(19) United States
(12) Patent Application Publication
(10) Pub. No.: US 2026/0317812 A1
(43) Pub. Date: Oct. 15, 2026

(54) IN-VITRO METHOD FOR DETECTING AT LEAST ONE NUCLEIC ACID, WHICH IS LOCATED OUTSIDE THE BLOOD CELLS IN WHOLE BLOOD IN THE CASE OF A LIVING BEING, AND DEVICE AND KIT FOR THIS PURPOSE

(71) Applicants: FRAUNHOFER-GESELLSCHAFT ZUR FÖRDERUNG DER ANGEWANDTEN FORSCHUNG E.V., München (DE); JOHANNES GUTENBERG-UNIVERSITÄT MAINZ, Mainz (DE)

(72) Inventors: Ralf HIMMELREICH, Mainz (DE); Perikles SIMON, Mainz (DE)

(73) Assignees: FRAUNHOFER-GESELLSCHAFT ZUR FÖRDERUNG DER ANGEWANDTEN FORSCHUNG E.V., München (DE); JOHANNES GUTENBERG-UNIVERSITÄT MAINZ, Mainz (DE)

(21) Appl. No.: 19/345,928

(22) Filed: Sep. 30, 2025

Related U.S. Application Data
(62) Division of application No. 17/414,146, filed on Dec. 18, 2019, now abandoned.

(30) Foreign Application Priority Data
Dec. 19, 2018 (DE) 10 2018 222 357.2

(51) Int. Cl. C12Q 1/6827; C12Q 1/6816; C12Q 1/6844; G01N 33/543

(52) U.S. Cl. CPC C12Q 1/6827; C12Q 1/6816; C12Q 1/6844; G01N 33/54388

(57) ABSTRACT

Disclosed is an in-vitro method, a device and a kit for detecting at least one nucleic acid which is located outside the blood cells in whole blood in the case of a living being. The advantage of the method is that it requires less time and equipment that known methods from the prior art, and that the risk of contamination and the risk of loss of cell-free nucleic acid to be amplified is minimised. The method is therefore more economical and has a higher detection accuracy than known detection methods for cell-free nucleic acids. In addition, the detection sensitivity is increased relative to detection methods from the prior art, which are based on a dilution of the blood. Also disclosed are the uses of the device and the kit.

### CROSS-REFERENCE TO RELATED APPLICATIONS

This patent application is a divisional of U.S. patent application Ser. No. 17/414,146, filed Jun. 15, 2021, which is the U.S. national phase of International Application No. PCT/EP2019/086122, filed on Dec. 18, 2019, which claims the benefit of German Patent Application No. 10 2018 222 357.2, filed Dec. 19, 2018, the disclosures of which are incorporated herein by reference in their entireties for all purposes.

An in vitro method, a device and a kit for detecting at least one nucleic acid (preferably DNA and/or RNA, particularly preferably DNA) that is located outside the blood cells in whole blood of a living being is provided. The advantage of the method in accordance with the invention is that the carrying out of the method requires less time effort and apparatus effort than known methods from the prior art and that the risk of a contamination, and of a loss of cell-free nucleic acid (e.g. cell-free DNA) to be amplified, is minimized. The method is therefore more economical and has a higher detection accuracy than known detection methods for cell-free nucleic acid (such as cell-free DNA). Furthermore, the detection sensitivity is above all increased over detection methods from the prior art that are based on a dilution of whole blood. A use of the provided device and of the provided kit is furthermore proposed.

In the whole blood of living beings, nucleic acid (e.g. DNA and/or RNA) can be present that is located outside the blood cells or not enclosed in a biological double membrane in the whole blood and thus so-to-say circulates “freely” in the blood of the living being. This co-called cell-free nucleic acid, which can, for example, be cell-free DNA (“cfDNA”) has a variable concentration in the blood plasma of approximately 1-1500 ng/ml. It is produced by dying cells, but for example also tumor cells and fetal cells release cfDNA (e.g. over the placenta in fetal cells). cfDNA is double stranded and predominantly has a size in the range from approximately 60 to 300 base pairs (“base pairs” is abbreviated to “bp” in the following). cfDNA frequently has a length of 140 bp because that approximately corresponds to the DNA portion of a nucleosome.

Freely circulating DNA currently forms the focus of many oncological problems. Increased concentrations in the blood (blood plasma or blood serum) correlate with the clinical stages in the acute phase of cancer development. The somatic cfDNA then still contains a portion of tumor cfDNA that is also called “ctDNA”.

The determination of ctDNA in cancer patients as part of a so-called “liquid biopsy” enables the monitoring of the therapy and will permit therapeutic measures in the future (so-called “precision medicine”).

An increased amount of freely circulating DNA in the blood plasma also occurs in a plurality of diseases (e.g. in autoimmune diseases, heart attack, strokes, and sepsis) and can also be observed on excessive muscle strain. Since the cfDNA acutely released on physical strain correlates to a high degree with pro-inflammatory immune markers, and mainly originates from immune cells, the cfDNA is a significant pro-inflammatory marker of so-called aseptic inflammation that is produced on physical strain. In this respect, the amount of released cfDNA is a marker for the degree of inflammation both on physical strain and on an inflammation reaction that accompanies different diseases.

A simple and fast method for quantifying cell-free nucleic acid, such as cfDNA, from a blood sample would be of great interest and very helpful for various medical problems. On an ambiguous result, such a method could provide an indication of the direction in which further diagnostic test methods are sensible.

The detection of cell-free nucleic acid, such as cell-free DNA, from blood has previously been done via an acquisition of blood plasma from a whole blood sample, a subsequent nucleic acid purification, and finally a nucleic acid amplification by means of a polymerase chain reaction (“PCR”). Alternatively, blood plasma has first been acquired from a whole blood sample, the blood plasma diluted, and finally a direct amplification has been carried out by means of PCR.

These detection methods known from the prior art have the disadvantage that they comprise laborious preparation steps for the preparation of the whole blood before the actual amplification reaction, i.e. the detection reaction of the cfDNA, is started. For example, in the known methods, blood plasma is first acquired from whole blood in a time-intensive manner, i.e. blood cells present in the whole blood are separated from cell-free whole blood. This is followed either by a nucleic acid purification or a dilution step.

The nucleic acid purification is very time-intensive. The alternative of diluting the blood plasma is admittedly faster, but due to the dilution of the sample necessarily suffers from a deterioration of the detection threshold (i.e. a lower detection sensitivity is to be expected). Methods from the prior art additionally use PCR, which requires a very long time from the start to the finish (often some hours) for the amplification and a comparatively large amount of knowledge and patience in the sample preparation.

The known detection processes are therefore all associated with a high time effort and a plurality of devices is required for the detection (e.g. a centrifuge and a PCR device). Some of the devices (e.g. the PCR device) can only be operated by trained personnel. Furthermore, a loss of material can occur in any of the required processing steps in the detection methods of the prior art and, due to the plurality and duration of the steps, the likelihood that the sample is contaminated with foreign DNA and that false positive results are obtained in the detection method is increased. In addition, lysis of blood cells cannot even be precluded in the step of acquiring blood plasma from the whole blood sample. The consequence is that there is also a risk of a contamination of the sample with own DNA that was present in the blood cells before the taking of the sample (in vivo) and was not present in the blood outside the blood cells, and in this case said own DNA causes false positive results in the measurement process.

The processes known from the prior art are characterized overall by a high work effort and device effort and a low detection accuracy (due to the increased risk of contamination). In processes that are based on a dilution of the sample, a low detection sensitivity is necessarily present.

Starting from this, it was the object of the present invention to provide a method and a device and a kit for carrying out the method that do not have the above-named disadvantages of the prior art. The method, the device, and the kit should in particular enable the detection of at least one nucleic acid (e.g. DNA and/or RNA) that is outside the blood cells in the whole blood of a living being with a smaller work effort, a smaller device effort, a higher detection accuracy, and a detection sensitivity that is as high as possible.

The object is achieved by the method, the device and/or the kit disclosed herein.

An in vitro method for detecting at least one nucleic acid (preferably DNA and/or RNA, particularly preferably DNA) which is located outside the blood cells in whole blood of a living being is provided in accordance with the invention. The method comprises the steps
a) providing a reaction mixture that is, together with at least one primer pair, suitable for the carrying out an isothermal amplification reaction, wherein the reaction mixture contains at least one primer pair that is suitable for the amplification of at least one nucleic acid (preferably DNA and/or RNA, particularly preferably DNA) present in whole blood, or at least one such primer pair is added to the reaction mixture;
b) adding whole blood of a living being to the reaction mixture;
c) carrying out an isothermal amplification reaction at a temperature in the range of ≤45° C.; and
d) Detecting DNA with the aid of at least one detection reagent, wherein the detection is carried out during step c) or after step c),
characterized in that the reaction mixture is suitable for preventing lysis of blood cells that are contained in the whole blood of the living being.

In accordance with the invention, the term “nucleic acid” is preferably understood as DNA and/or RNA, particularly preferably DNA. Consequently, the nucleic acid to be detected or the nucleic acid to be amplified can, for example, be DNA and/or RNA. The term “nucleic acid” preferably also covers RNA-DNA hybrids. Such RNA-DNA hybrids are, for example, individual, short DNA and RNA strands (in particular strands of a length of 20-22 nucleotides) that have attached to one another.

If the nucleic acid to be detected comprises or consists of RNA, the reaction mixture preferably comprises an enzyme, or an enzyme is added to it, that can copy RNA into DNA (e.g. the enzyme reverse transcriptase).

The reaction mixture can comprise proteins from the group of nucleic acid ligases (e.g. a T7 RNA polymerase) or proteins from the group of nucleic acid ligases can be added to it.

An isothermal amplification of free RNA or of RNA present within an RNA-DNA hybrid can be achieved, for example, by a NASBA process. In this process, the RNA is amplified at a constant temperature (e.g. room temperature, 25° C.) in the reaction mixture (with the aid of at least one primer that comprises a T7 promoter sequence) via an interaction of the enzymes RNAse H, reverse transcriptase, SD polymerase, and T7 RNA polymerase. It is consequently preferred that the reaction mixture comprises these enzymes and at least one primer with a T7 promoter sequence or that these enzymes and this primer are added to it.

Alternatively or additionally, an isothermal amplification of free RNA, or of RNA present in an RNA-DNA hybrid, can be achieved via a modification of the NASBA process that is called a “linker ligation” NASBA process. In this process, the RNA is amplified at a constant temperature (e.g. room temperature, 25° C.) in the reaction mixture (with the aid of at least one primer that comprises a T7 promoter sequence) via an interaction of the enzymes poly (A) polymerase, RNAse H, and T7 RNA polymerase. It is preferred in this modification that the reaction mixture comprises these enzymes and at least one primer with a T7 promoter sequence or that these enzymes and this primer are added to it.

The above-named NASBA process can also be carried out in combination with an RPA process. In this process, DNA present in the whole blood is first converted into RNA by the RPA process (NASBA amplification), with the RNA then being amplified by the NASBA process.

It has been found that, with the method according to the invention, the detection of at least one nucleic acid (e.g. DNA and/or RNA) that is present outside the blood cells in the whole blood of a living being (with DNA as a nucleic acid, so-called cell-free DNA or in abbreviated from as cfDNA) is also possible when not, as customary in the prior art, a purified blood sample is used, but when whole blood is used directly, i.e. without pretreatment. The requirement for this is that the reaction mixture is suitable for preventing lysis of blood cells that are contained in the whole blood of the living being. It can be prevented by a use of such a reaction mixture that nucleic acids (e.g. DNA and/or RNA) that are present in blood cells containing cell nuclei (e.g. leukocytes) negatively influence the detection process, i.e. deliver false positive results.

In a preferred embodiment of the method in accordance with the invention, the reaction mixture is generally suitable for preventing lysis of compartments that contain nucleic acids and are surrounded by a lipid double membrane, and that are contained in the whole blood of the living being. The term “compartments containing nucleic acid surrounded by a lipid double membrane” is broadly understood in accordance with the invention, i.e. it comprises all the conceivable compartments that contain nucleic acid and that are surrounded by a biological lipid double membrane. They, for example, include mitochondria, extracellular vesicles (exosomes and microparticles), peroxisomes, viruses, single-celled organisms, and bacteria.

The advantage of the method in accordance with the invention in comparison with known methods is that a sample preparation, i.e. a purification of the whole blood, is not necessary. The method in accordance with the invention therefore manages with fewer method steps and can be carried out faster.

Furthermore, the apparatus effort required in known methods for the purification of the whole blood, i.e. for the preparation of the sample, is not necessary. There is thus a considerable cost advantage over known methods.

In addition, the method in accordance with the invention is exceptionally suitable for implementation outside of a laboratory. This means that a patient can determine ambulatory (i.e. at his home using a device suitable for this purpose or using a kit suitable for this purpose for carrying out the method) whether at least one cell-free nucleic acid (e.g. cfDNA) is generally present in his blood (use of non-specific primers) or whether at least one specific cell-free nucleic acid (e.g. a specific cfDNA) is present in his blood (use of specific primers). The tested person can be an athlete and/or a patient, such as an accident patient or an emergency patient, who can be examined directly after the sport or directly after an accident or a medical emergency directly on site as to whether at least one (specific) nucleic acid (e.g. cfDNA) or an increased amount of at least one (specific) nucleic acid (e.g. cfDNA) is present in his blood circulation. The obtained information can represent a significant advantage to speedily trigger an adequate treatment of a patient.

There is furthermore generally a risk in every method step, i.e. also in the method steps typical in the prior art for the purification of the whole blood sample, that nucleic acid (e.g. DNA) to be detected is lost (e.g. DNA and/or RNA can adsorb at specific surfaces) and can thus no longer be detected in the actual detection process. The consequence is a low detection sensitivity or false negative results. The risk of losing nucleic acid (e.g. DNA and/or RNA) to be detected on the way to detection is reduced by the method in accordance with the invention since whole blood is directly used. The detection sensitivity and detection accuracy are consequently higher than in the known processes. The method in accordance with the invention is suitable for detecting so-called low copy number genes or even so-called single copy number genes in whole blood.

Unlike by the steps for the purification of the whole blood sample in the known methods from the prior art, there is also no risk in the method in accordance with the invention that the sample is contaminated by foreign nucleic acids (e.g. DNA of the person performing the method or bacterial DNA) during one of these steps. Consequently, by the method in accordance with the invention, the risk of false positive signals is curtailed, whereby the detection accuracy over the known processes is further increased.

The method in accordance with the invention can be characterized in that that reaction mixture forms a liquid mixture with the whole blood, wherein the liquid mixture is substantially isotonic with blood cells.

The reaction mixture can furthermore form a liquid mixture with the whole blood that has an osmolarity that substantially corresponds to the osmolarity of blood cells, preferably an osmolarity in the range from 230 to 350 mosmol/kg, preferably 260 to 320 mosmol/kg, particularly preferably 270 to 310 mosmol/kg, in particular 280 to 300 mosmol/kg.

In a preferred embodiment, the reaction mixture does not comprise any surfactants in concentrations that are suitable for effecting lysis of blood cells, preferably no substances or substance mixtures that are suitable for effecting lysis of blood cells. It is in particular understood by this that the reaction mixture does not contain any surfactants, or any substances or substance mixtures, in a concentration that is suitable to effect lysis of blood cells. This feature optionally does not only relate to blood cells, but rather also generally to compartments containing nucleic acid surrounded by a lipid double membrane that are contained in the whole blood of the living being. As already mentioned above, the term “compartments containing nucleic acid surrounded by a lipid double membrane” is broadly understood here, i.e. it comprises all the conceivable compartments that contain nucleic acid and that are surrounded by a biological double membrane. This includes, for example, mitochondria, exosomes, single-cell organisms, and bacteria.

The reaction mixture used in the method can contain at least one of the following substances or at least one of the following substances can be added to the reaction mixture:
i) nucleotide triphosphates, preferably dATP, dCTP, dGTP, dTTP, ATP, GTP; and/or
ii) creatine kinase, and phosphocreatine; and/or
iii) a magnesium salt, preferably magnesium acetate; and/or
iv) polyethylene glycol.

The reaction mixture can furthermore contain at least one of the following proteins or at least one of the following proteins can be added to the reaction mixture:
i) a strand displacing polymerase, preferably selected from the group consisting of Sau DNA polymerase, Bsu DNA polymerase, Klenow fragment, phi29, and combinations thereof; and/or
ii) a recombinase, preferably RecA recombinase and/or T4 UvsX; and/or
iii) a single strand binding protein, preferably SSB and/or T4 gp32; and/or
iv) an exonuclease, preferably exonuclease III and/or exonuclease IV.

In a preferred embodiment, the detection reagent used in the method is not suitable for passing through the cell membrane of blood cells that are contained in the whole blood. This feature can in particular not only apply to blood cells, but generally to the membrane of compartments containing nucleic acid surrounded by a lipid double membrane. It is advantageous in this embodiment that the detection of amplified nucleic acid (e.g. DNA) can also be carried out without any prior separation of cells comprising DNA or of the above-named compartments since there is no risk of obtaining false positive results by binding the detection reagent to DNA within the cells or within the above-named compartments. The method can therefore be carried out more simply and faster (e.g. no step is required to allow the reaction mixture to flow from a reaction space over a dividing wall to a separate detection space).

The detection reagent can be suitable for detecting single-strand and/or double-strand DNA and used therefor.

The detection reagent can be selected from the group consisting of probe with a fluorophore, DNA binding dye molecule, reagent for the detection of pyrophosphate, and combinations thereof.

The probe with fluorophore can comprise or consist of an oligonucleotide that comprises or consists of a quencher, a fluorophore, and a DNA sequence present between the quencher and the fluorophore, wherein the DNA sequence is suitable (by an internal modification) for being split by an endonuclease or an exonuclease and preferably is a DHF or AP-DNA sequence. The dye molecule-binding DNA can be selected from the group consisting of EVAGreen, SYBR Green, Syto dyes, TOPO dyes, and combinations thereof. Preferably, for a particularly sensitive detection, dye molecules that have absorption and emission spectra that differ (preferably considerably) from the corresponding spectra of a whole blood sample are used. With respect to the absorption spectrum of the dye molecule used, an absorption spectrum of less than 525 nm, one between 545 nm and 570 nm, or one above 590 nm, is particularly advantageous since relative local absorption minima of the whole blood are present in these ranges. In other words, the dye should not have a (high) absorption at a wavelength of 525 nm to 545 nm and/or at a wavelength of 570 to 590 nm. An emission spectrum between 580 nm and 610 nm, in particular 590 nm and 610 nm, is particularly advantageous for the emission. The reagent for the detection of pyrophosphate can be a magnesium salt.

In a preferred embodiment of the method, the detection of DNA takes place via a detection unit, wherein the detection unit preferably is selected from the group consisting of an absorption measurement device, a fluorescence measurement device, a turbidimeter, a camera, a cellphone (e.g. internal camera of the cellphone, optionally in conjunction with evaluation software on the cellphone), and combinations thereof.

Furthermore, a quantification of the amount of amplified DNA can take place in the method via an evaluation unit, wherein the evaluation unit preferably is communicatively connected to a detection unit (preferably to an above-named detection unit).

In the method, at least one primer, optionally both primers of the primer pair, can have a length of 20 to 45 bp, particularly preferably 25 to 40 bp, in particular 30 to 35 bp.

In addition, at least one primer of the primer pair, optionally both primers of the primer pair, can be suitable for binding to a repetitive DNA sequence in the DNA of the living being, preferably to a repetitive sequence selected from the group consisting of a LINE sequence, SINE sequence, and ALU sequence.

The advantage of the use of primers or primer pairs that bind to repetitive sequences is that they are suitable for the unspecific amplification of all the conceivable unknown DNAs that occur in the whole blood of living beings and include repetitive DNA sequences. Since practically every DNA or every DNA fragment has a certain length of repetitive DNA sequences, it is possible via such primers or primer pairs to amplify every conceivable DNA that is present in the whole blood. In other words, the sequence of the DNA to be amplified does not have to be known prior to the amplification reaction. The method can therefore generally deliver the proof whether free DNA is present at all in the blood of the living being.

If free DNA is actually detected in the whole blood via the method, this can of course be sequenced using common methods. If the specific sequence of the free DNA (or of the free DNAs) in the blood of a certain living being (e.g. of a specific patient) is known, the method in accordance with the invention can of course also be carried out with primer pairs that are directly suitable for detecting the known DNA(s). A monitoring of specific free DNAs in the whole blood of a patient can thus be carried out over a specific time period (e.g. minutes, hours, days, and/or months) with the method in accordance with the invention.

A primer of the primer pair, optionally both primers of the primer pair, can be suitable for binding to a DNA sequence in the DNA of the living being that is present in the blood circuit at the start, during, or after a disease and/or at the start, during, or after an excessive muscle strain of the living being.

The living being can be a human or an animal.

The isothermal DNA amplification reaction used in the method can be carried out at a temperature in the range from 20° C. to 44° C., preferably 25° C. to 42° C., particularly preferably 30° C. to 41° C., in particular 35° C. bis 40° C. The temperature required for this purpose can be produced or set by a temperature control unit.

If no temperature control unit is used in the method, the reaction can either take place at room temperature or the required reaction temperature can be produced via body heat (of a human or animal). The latter is possible, for example, in that the reaction mixture of the device is moved so close to a body of a human or animal that a heat transfer is possible from the body to the reaction mixture.

The isothermal amplification reaction can be a semiquantitative isothermal DNA amplification reaction or a quantitative isothermal DNA amplification reaction.

The isothermal DNA amplification reaction can be an amplification reaction that is selected from the group consisting of recombinase polymerase amplification reaction, Siba HDA, NASBA, and SDA amplification reaction; it is optionally selected from the group consisting of recombinase polymerase amplification reaction, Siba, and SDA amplification reaction. It is preferably a recombinase polymerase amplification reaction. The reaction mixture for the amplification reaction optionally includes a helicase. A preferred temperature for the amplification is 37° C., i.e. the reaction mixture is preferably temperature controlled to a temperature of 37° C.

During or after step d) of the method in accordance with the invention, in the case of (detected) amplified DNA, a conclusion can be drawn on the presence of a disease and/or of an excessive muscle strain. The disease is in particular selected from the group consisting of inflammation, cancer, autoimmune disease, coronary, stroke, sepsis, chromosomal aberration, change in copy number in genes, gene mutation, and combinations thereof.

The reaction mixture used can comprise a plurality of primer pairs, optionally at least 2, 3, 4, 5, 6, 7, 8, 9, or 10 primer pairs, that are each suitable for amplification of at least one nucleic acid (e.g. DNA) present in the whole blood. Alternatively or additionally, a plurality of such primer pairs can be added to the reaction mixture, wherein the respective primer pairs are preferably suitable for amplifying a different nucleic acid (e.g. DNA), preferably a nucleic acid (e.g. DNA), that is present in the whole blood of a living being on excessive muscle strain and/or in a disease selected from the group consisting of inflammation, cancer, autoimmune disease, coronary, stroke, sepsis, chromosomal aberration, change of copy number in genes, gene mutation, and combinations thereof.

In a preferred embodiment, the reaction mixture and the detection reagent, optionally the reaction mixture, the primer pair, and the detection reagent, are contained by a lateral flow strip.

In accordance with the invention, a device and/or a kit is/are provided that is suitable for in vitro detection of at least one nucleic acid (preferably DNA and/or RNA, particularly preferably DNA) which is located outside the blood cells in whole blood in a living being. The device and/or the kit comprises/comprise
a) a reaction mixture that is, together with at least one primer pair, suitable for the carrying out of an isothermal amplification reaction at a temperature in the range of ≤45° C.;
b) at least one primer pair that is suitable for the amplification of at least one nucleic acid present in the whole blood (preferably DNA and/or RNA, particularly preferably DNA), with the at least one primer pair optionally being contained in the reaction mixture;
c) at least one detection reagent for the detection of DNA, with the at least one detection reagent optionally being contained in the reaction mixture,
characterized in that the reaction mixture is suitable for preventing lysis of blood cells that are contained in the whole blood in a living being.

The advantages named above in connection with the method in accordance with the invention apply accordingly to the device in accordance with the invention and the kit in accordance with the invention. The device in accordance with the invention can have the features named above in connection with the method in accordance with the invention or can be configured for their performance.

If the nucleic acid to be detected comprises or consists of RNA, the reaction mixture preferably comprises an enzyme that can copy RNA into DNA (e.g. the reverse transcriptase enzyme).

The reaction mixture can contain proteins from the group of nucleic acid ligases (e.g. a T7 RNA polymerase).

The reaction mixture preferably contains RNAse H, reverse transcriptase, SD polymerase, T7 RNA polymerase, and at least one primer having a T7 promoter sequence.

The reaction mixture can also preferably contain the enzymes poly (A) polymerase, RNAse, T7 RNA polymerase, and at least one primer having a T7 promoter sequence.

The reaction mixture contained in the device and/or in the kit can be suitable for forming a liquid mixture with whole blood that is substantially isotonic with blood cells.

The reaction mixture can furthermore be suitable for forming a liquid mixture with the whole blood that has an osmolarity that substantially corresponds to the osmolarity of blood cells, preferably an osmolarity in the range from 230 to 350 mosmol/kg, preferably 260 to 320 mosmol/kg, particularly preferably 270 to 310 mosmol/kg, in particular 280 to 300 mosmol/kg.

In a preferred embodiment of the device and/or of the kit, the device and/or the kit does/do not comprise any surfactants that are suitable for effecting lysis of blood cells, preferably no substances or substance mixtures that are suitable for effecting lysis of blood cells. It is in particular understood by this that the reaction mixture does not contain any surfactants or any substances or substance mixtures in a concentration that is suitable to effect lysis of blood cells. This feature optionally does not only relate to blood cells, but rather also generally to compartments containing nucleic acid surrounded by a lipid double membrane that are contained in the whole blood in the living being. As already mentioned above, the term “compartments containing nucleic acid surrounded by a lipid double membrane” is broadly understood here, i.e. it comprises all the conceivable compartments that contain nucleic acid and that are surrounded by a biological double membrane. This includes, for example, mitochondria, exosomes, single-cell organisms, and bacteria.

The reaction mixture can comprise at least one of the following substances:
i) nucleotide triphosphates, preferably dATP, dCTP, dGTP, dTTP, ATP, GTP; and/or
ii) creatine kinase, and phosphocreatine; and/or
iii) a magnesium salt, preferably magnesium acetate; and/or
iv) polyethylene glycol.

The reaction mixture can furthermore comprise at least one of the following proteins:
i) a strand displacing polymerase, preferably selected from the group consisting of Sau DNA polymerase, Bsu DNA polymerase, Klenow fragment, phi29, and combinations thereof; and/or
ii) a recombinase, preferably RecA recombinase and/or T4 UvsX; and/or
iii) a single strand binding protein, preferably SSB and/or T4 gp32; and/or
iv) an exonuclease, preferably exonuclease Ill and/or exonuclease IV.

In a preferred embodiment, the detection reagent contained in the device and/or in the kit is not suitable for passing through the cell membrane of blood cells that are contained in the whole blood. This feature can in particular not only apply to blood cells, but also generally to the lipid double membrane of compartments containing nucleic acid surrounded by a lipid double membrane. It is advantageous in this embodiment that the detection of amplified DNA can also be carried out without any prior separation of cells comprising DNA or the above-named compartments since there is no risk of obtaining false positive results by binding the detection reagent to DNA within the cells or within the above-named compartments. The device and/or the kit can therefore be held in smaller sizes (e.g. no reaction space and no separate detection space having a dividing wall disposed therebetween are necessary).

The detection reagent can be suitable for the detection of single-strand and/or double-strand DNA.

Furthermore, the detection reagent of the device and/or of the kit can be selected from the group consisting of a probe with fluorophore, DNA binding dye molecule, rreagent for the detection of pyrophosphate, and combinations thereof.

The probe with fluorophore can comprise or consist of an oligonucleotide that comprises or consists of a quencher, a fluorophore, and a DNA sequence present between the quencher and the fluorophore, with the DNA sequence being suitable for being split by an endonuclease or an exonuclease and preferably being a DHF or AP-DNA sequence. The dye molecule binding DNA can be selected from the group consisting of EVAGreen, SYBR Green, Syto dyes, TOPO dyes, and combinations thereof. For a particularly sensitive detection, dye molecules that have absorption and emission spectra that differ (preferably considerably) from the corresponding spectra of a whole blood sample are preferably used. With respect to the absorption spectrum of the dye molecule used, an absorption spectrum of less than 525 nm, one between 545 nm and 570 nm, or one above 590 nm is particularly advantageous since relative local absorption minima of the whole blood are present in these ranges. In other words, the dye should not have a (high) absorption at a wavelength of 525 nm to 545 nm and/or at a wavelength of 570 to 590 nm. An emission spectrum between 580 nm and 610 nm, in particular 590 nm and 610 nm is particularly advantageous for the emission. The reagent for the detection of pyrophosphate can be a magnesium salt.

In a preferred embodiment, the device and/or the kit contains/contain a detection unit for the detection of DNA, with the detection unit preferably being selected from the group consisting of an absorption measurement device, a fluorescence measurement device, a turbidimeter, a camera, a cellphone (e.g. internal camera of the cellphone, optionally in conjunction with evaluation software on the cellphone), and combinations thereof.

The device and/or the kit can furthermore contain an evaluation unit for the quantification of an amount of amplified DNA, with the evaluation unit preferably being communicatively connected to a detection unit.

At least one primer of the primer pair, optionally both primers of the primer pair, can have a length of 20 to 45 bp, particularly preferably 25 to 40 bp, in particular 30 to 35 bp.

Furthermore, a primer of the primer pair, optionally both primers of the primer pair, can be suitable for binding to a repetitive DNA sequence in the DNA of the living being, preferably to a repetitive sequence selected from the group consisting of a LINE sequence, SINE sequence, and ALU sequence.

In addition, at least one primer of the primer pair, optionally both primers of the primer pair, can be suitable for binding to a DNA sequence in the DNA of the living being that is present in the blood circuit at the start, during, or after a disease and/or at the start, during, or after an excessive muscle strain of the living being.

The living being can be a human or an animal.

The device and/or the kit can include a temperature control unit that is configured to regulate the reaction mixture to a temperature in the range from ≤45° C., preferably to a temperature in the range from 20° C. to 44° C., particularly preferably 25° C. to 42° C., particularly preferably 30° C. to 41° C., in particular 35° C. to 40° C. The temperature control unit is preferably supplied with energy from a voltage source that has a voltage in the range from 1 to 12 V.

It is, however, also conceivable that the device and/or the kit does/do not contain any temperature control unit. The reaction can then take place either at room temperature or be generated via body heat (of a human or of an animal). The latter is possible, for example, in that the reaction mixture of the device is moved so close to a body of a human or animal that a heat transfer is possible from the body to the reaction mixture.

The isothermal amplification reaction can be a semiquantitative isothermal DNA amplification reaction or a quantitative isothermal DNA amplification reaction.

Furthermore, the isothermal DNA amplification reaction can be an amplification reaction that is selected from the group consisting of recombinase polymerase amplification reaction, Siba HDA, NASBA, and SDA amplification reaction; it is optionally selected from the group consisting of recombinase polymerase amplification reaction, Siba, and SDA amplification reaction. It is preferably a recombinase polymerase amplification reaction. The reaction mixture for the amplification reaction optionally includes a helicase. A preferred temperature for the amplification is 37° C., i.e. the reaction mixture is preferably temperature controlled to a temperature of 37° C.

The device and/or kit can be configured to display, in the event of a detection of DNA by the detection reagent, the presence of a disease and/or of an excessive muscle strain, optionally via a boundary of the device transparent for visible light. The disease is in particular selected from the group consisting of inflammation, cancer, autoimmune disease, coronary, stroke, sepsis, chromosomal aberration, change in copy number in genes, gene mutation, and combinations thereof.

In a preferred embodiment, the device and/or the kit contains/contain a plurality of primer pairs, optionally at least 2, 3, 4, 5, 6, 7, 8, 9, or 10 primer pairs, with each primer pair being suitable for amplifying at least one nucleic acid (e.g. DNA) present in the whole blood, and with the respective primer pairs preferably being suitable for amplifying a different nucleic acid (e.g. DNA), preferably a nucleic acid (e.g. DNA), that is present in the whole blood of a living being on an excessive muscle strain and/or in a disease selected from the group consisting of inflammation, cancer, autoimmune disease, coronary, stroke, sepsis, chromosomal aberration, change of copy number in genes, gene mutation, and combinations thereof.

The device is preferably a lateral flow strip.

The kit is preferably characterized in that it has a lateral flow strip that includes the reaction mixture; or includes the reaction mixture and the detection reagent.

In addition, the use of the device in accordance with the invention and/or of the kit in accordance with the invention is proposed for the direct, semiquantitative or quantitative, detection of at least one nucleic acid (e.g. DNA and/or RNA) in the whole blood, preferably for the direct detection of at least one nucleic acid (e.g. DNA and/or RNA) that is present outside the blood cells in the whole blood of the living being on a disease of a living being and/or on an excessive muscle strain.

### BRIEF DESCRIPTION OF THE DRAWINGS

The subject matter in accordance with the invention will be explained in more detail with reference to the following Figures and examples without intending to restrict it to the specific embodiments shown here.

FIG. 1 shows the result of a quantitative isothermal amplification reaction of a specific cfDNA via a probe with a direct use of whole blood (see Example 2). It can be seen from the amplification plot that the detection by the probe also works on the direct use of (non-purified) whole blood. It furthermore becomes clear that an increase of the cfDNA can be detected due to training in the trial person from whom the whole blood comes.

FIG. 2 shows the result of a quantitative isothermal amplification reaction of a specific cfDNA via a DNA intercalating fluorescent dye with a direct use of whole blood (see Example 4). It can be seen from the amplification plot that the detection by the fluorescent dye also works on the direct use of (non-purified) whole blood. It furthermore becomes clear that an increase of the cfDNA can be detected due to training in the trial person from whom the whole blood comes.

FIG. 3 shows the result of a quantitative isothermal amplification reaction of a specific cfDNA via a probe with a direct use of human gDNA that has been contaminated with different amounts of hemolysate (see Example 3). It can be seen from the amplification plot that hemolysate exerts a significantly inhibiting effect on the amplification reaction that only disappears from a strong dilation of 1:10,000 (v/v) with water onward.

### DETAILED DESCRIPTION OF THE INVENTION

### Example 1—Detection of a Cell-Free DNA Via a Probe with a Direct Use of Whole Blood

An isothermal amplification reaction was carried out at a temperature of 40° C. for the amplification of at least one specific cell-free DNA using a sample of whole blood that contains cell-free DNA (so-called cfDNA). The amplified DNA was detected via a probe.

The following protocol was used:
preparing the rehydration mixture (total volume: 63.08 μl)
6.72 μl primer mixture of forward primer (“301” primer) and reverse primer (“302” primer) for the detection of cfDNA that enters into the blood circulation (per 10 μM) on muscle strain;
0.96 μl probe (10 μM);
47.2 μl rehydration buffer (from “TwistAmp® exo” kit, TwistDX Inc., Cambridge, USA) comprising tris buffer, potassium acetate, and PEG 35000, preferably 25 mM tris buffer, 100 mM potassium acetate, and 5.46% (w/v) PEG 35000 (see e.g. paragraph in WO 2010/141940 A1);
8.2 μl H₂O.
mixing;
resuspending and dissolving a freeze dried pellet of a “TwistAmp®exo” kit (TwistDX Inc., Cambridge, USA) with 63 μl rehydration mixture (by pipetting up and off);
mixing;
transferring a respective 19.6 μl of the dissolved pellet into an 0.5 ml Eppendorf cup;
adding 1 μm mouse plasma as a control into one of the Eppendorf cup and a respective 1 μl EDTA whole blood as a sample into the other Eppendorf cups;
mixing;
adding a respective 6.4 μm magnesium acetate solution (concentration: 80 mM);
mixing;
adding a respective 7 μl from each Eppendorf cup onto a PCR well plate;
mixing PCR well plate;
incubating the PRC well plate in the CFX Thermo Cycler at 40° C. (with an open lid, i.e. “LidOff”).

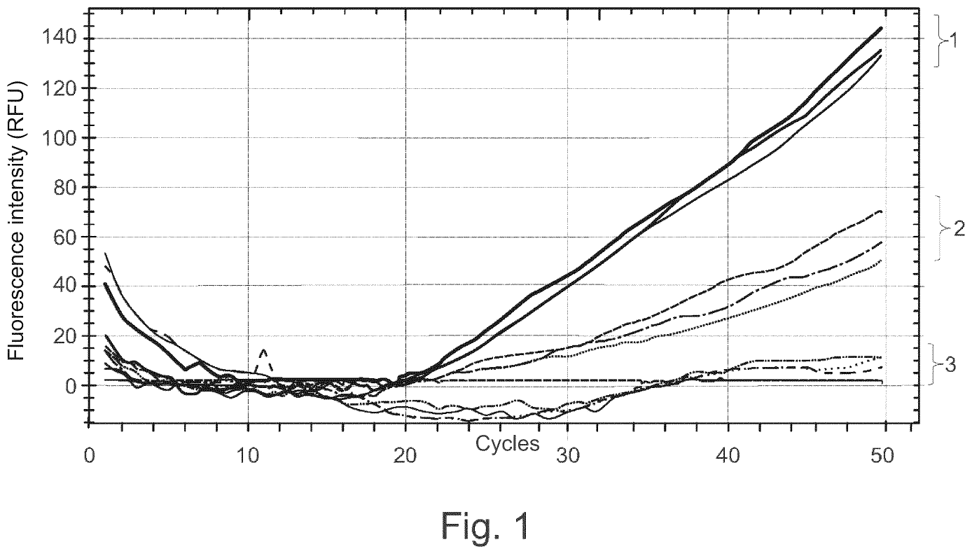

The result is shown in FIG. 1. It can be clearly recognized that the detection method in accordance with the invention using a probe as the detection reagent and a direct use of (non-purified) whole blood works. The concentration of cfDNA in the whole blood of the trial person before the training (see signal at 2) is smaller than after the training (see signal at 1). The control plasma sample from a mouse (NTC plasma) does not show any amplification of the target cfDNA (see signal at 3), which documents a specificity of the amplification reaction shown here for human cfDNA.

### Example 2—Detection of a Cell-Free DNA Via a Fluorescent Dye with a Direct Use of Whole Blood

An isothermal amplification reaction was carried out at a temperature of 40° C. for the amplification of at least one specific cell-free DNA using a sample of whole blood that contains cell-free DNA (so-called cfDNA). The amplified DNA was detected by a fluorescent dye (“EvaGreen® Dye”, Biotium Inc., Fremont, USA).

The following protocol was used:
preparing the rehydration mixture (total volume: 63.52 μl)
6.72 μl primer mixture of forward primer (“63” primer) and reverse primer (“66” primer) for the detection of cfDNA that enters into the blood circulation (per 10 μM) on muscle strain;
4 μl EvaGreen® Dye (20×) (Biotium Inc., Fremont, USA);
47.2 μl rehydration buffer (from “TwistAmp® exo” kit, TwistDX Inc., Cambridge, USA) comprising tris buffer, potassium acetate, and PEG 35000, preferably 25 mM tris buffer, 100 mM potassium acetate, and 5.46% (w/v) PEG 35000 (see e.g. paragraph in WO 2010/141940 A1);
5.6 μl H₂O.
mixing;
resuspending and dissolving a freeze dried pellet of a “TwistAmp®exo” kit (TwistDX Inc., Cambridge, USA) with 63 μl rehydration mixture (by pipetting up and off);
mixing;
transferring a respective 19.6 μl of the dissolved pellet into an 0.5 ml Eppendorf cup;
adding 1 μm mouse plasma as a control into one of the Eppendorf cup and a respective 1 μl EDTA whole blood as a sample into the other Eppendorf cups;
mixing;
adding a respective 6.4 μm magnesium acetate solution (concentration: 80 mM);
mixing;
adding a respective 7 μl from each Eppendorf cup onto a PCR well plate;
mixing PCR well plate;
incubating the PRC well plate in the CFX Thermo Cycler at 40° C. (with an open lid, i.e. “LidOff”).

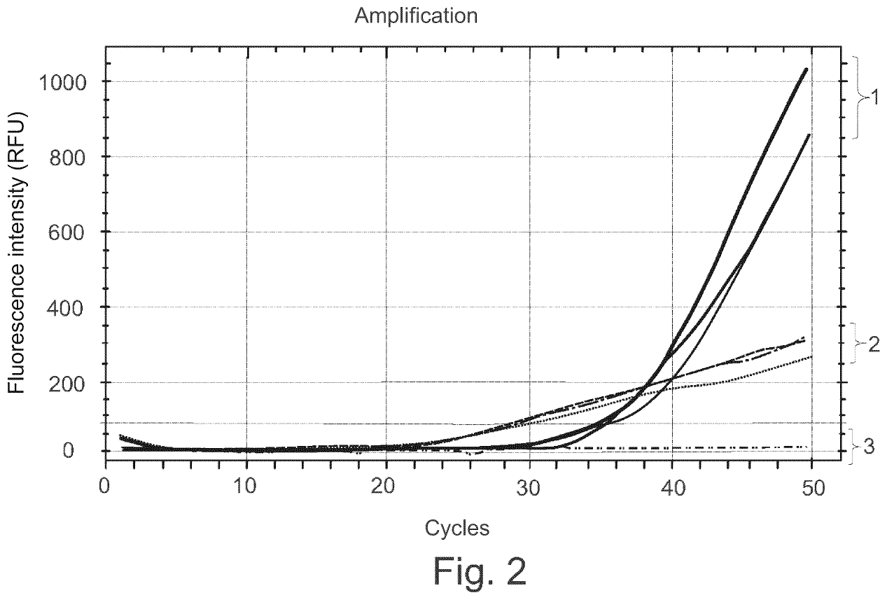

The result is shown in FIG. 2. It can be clearly recognized that the detection method in accordance with the invention using an intercalating fluorescent dye as the detection reagent and a direct use of (non-purified) whole blood works. The concentration of cfDNA in the whole blood of the trial person before the training (see signal at 2) is smaller than after the training (see signal at 1). The control plasma sample from a mouse (NTC plasma) does not show any amplification of the target cfDNA (see signal at 3), which documents a specificity of the amplification reaction shown here for human cfDNA.

### Example 3—Detection of a Lack of Lysis of Whole Blood During the Isothermal Amplification Reaction

A hemolysate is first prepared in that mouse blood is treated by a plurality of defrosting and freezing cycles, whereby lysis of the blood cells occurs.
Formulation: RPA probes for whole blood;
Primer: Forward primer (“301” primer) and reverse primer (“302” primer) for the detection of cfDNA that enters into the blood circulation on muscle strain;
Template: 1 μl human gDNA (50 ng/ml) with 1 μl water or with 1 μl undiluted hemolysate or a mixture of a different amount of lyzed whole blood (hemolysate) with water. Hemolysate was respectively diluted with water at 1:10 (v/v), 1:100 (v/v), 1:1000 (v/v), and 1:10,000 (v/v) for the mixture of hemolysate with water.

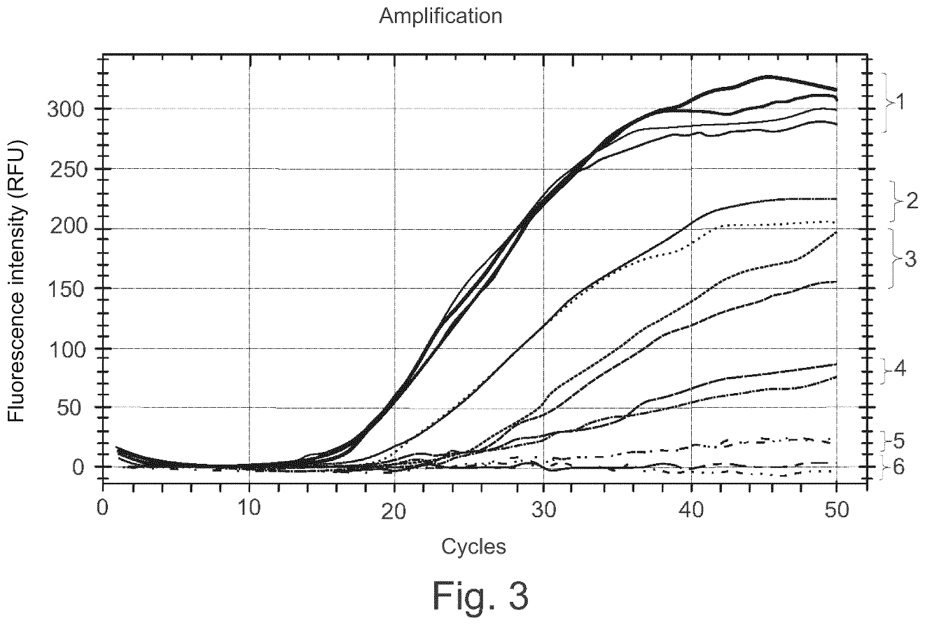

The result is shown in FIG. 3 and n Table 1. The sample with 1 μl human gDNA (50 ng/ml) and 1 μl water can be seen at 1. The samples with 1 μl human gDNa (50 ng/ml) and a differing amount of hemolysate can be seen at 2, 3, 4, 5, and 6. Here, the signal for hemolysate:water=1:10,000 (v/v) is shown at 2; for hemolysate:water=1:1000 (v/v) at 3; for hemolysate:water=1:100 (v/v) at 4; for hemolysate:water=1:10 (v/v) at 5; and for undiluted hemolysate at 6. It can be clearly recognized in FIG. 3 that an increasing amount of hemolysate in the reaction mixture during isothermal amplification reaction inhibits the amplification reaction of target cfDNA. Hemolysate portions in a 1:10,000 dilution already significantly impair the reaction kinetics (cf. signals at 1 and 2). The reaction kinetics that were obtained in the absence of hemolysate is comparable with the reaction kinetics in the method in accordance with the invention with whole blood of Examples 1 and 2. The respective obtained values of the fluorescent threshold cycle are compiled in Table 1 (see Table 1).

TABLE 1

| Template: | Fluorescent threshold cycle (Cq) |
|---|---|
| Undiluted hemolysate | ND |
| Hemolysate:water = 1:10 (v.v) | ND |
| Hemolysate:water = 1:100 (v.v) | 30.6 |
| Hemolysate:water = 1:1000 (v.v) | 24.1 |
| Hemolysate:water = 1:10,000 (v.v) | 19.4 |
| No hemolysate (only water) | 19.4 |

ND = not determinable

It can be concluded from these results that the blood cells in the whole blood are not destroyed during the isothermal amplification reaction in the method in accordance with the invention and thus no substances that inhibit it are released during the amplification reaction. A high detection sensitivity and detection accuracy can consequently also be ensured on the use of whole blood by the method in accordance with the invention.

1-24. (canceled)

25. An in vitro method for detecting at least one nucleic acid that is located outside the blood cells in whole blood of a living, the method comprising:
a) providing a reaction mixture that is, together with at least one primer pair, suitable for the carrying out an isothermal amplification reaction, wherein the reaction mixture contains at least one primer pair that is suitable for amplifying at least one nucleic acid present in whole blood, or at least one such primer pair is added to the reaction mixture;
b) adding whole blood of a living being, without carrying out a purification of said whole blood, to the reaction mixture;
c) carrying out an isothermal amplification reaction with the reaction mixture of step (b), which comprises said whole blood, at a temperature of ≤45° C.; and
d) detecting at least one nucleic acid with the aid of at least one detection reagent, wherein the detection is carried out during or after step (c),
wherein the reaction mixture is suitable for preventing lysis of blood cells that are contained in the whole blood of the living being,
wherein the reaction mixture is provided such that it does not comprise any substances or substance mixtures that are suitable for effecting lysis of blood cells, and
wherein the reaction mixture is provided such that it forms, in step (b), a liquid mixture with the whole blood that has an osmolarity that corresponds to the osmolarity of blood cells.

26. The method of claim 25, wherein the reaction mixture is provided such that it forms, in step (b), a liquid mixture with the whole blood that has an osmolarity in the range from 280 to 300 mosmol/kg.

27. The method of claim 25, wherein the reaction mixture is provided such that it forms, in step (b), a liquid mixture with the whole blood that is isotonic with blood cells.

28. The method of claim 25, wherein the detection reagent is not suitable for passing through the cell membrane of blood cells that are contained in the whole blood.

29. The method of claim 25, wherein the detection reagent is selected from the group consisting of probe with fluorophore, DNA binding dye molecule, reagent for the detection of pyrophosphate, and any combinations thereof.

30. The method of claim 25, wherein the detection of nucleic acid takes place by a detection unit.

31. The method of claim 25, wherein a quantification of the amount of amplified nucleic acid takes place by an evaluation unit.

32. The method of claim 25, wherein both primers of the primer pair are suitable for binding to a repetitive DNA sequence in the nucleic acid of the living being.

33. The method of claim 25, wherein both primers of the primer pair are suitable for binding to a DNA sequence in the DNA of the living being that is present in the blood circuit at the start, during, or after a disease and/or at the start, during, or after an excessive muscle strain of the living being.

34. The method of claim 25, wherein, in the case of amplified nucleic acid during or after step d), a conclusion is drawn on the presence of a disease and/or of excessive muscle strain.

35. The method of claim 25, wherein the reaction mixture contains a plurality of primer pairs that are each suitable for amplifying at least one nucleic acid present in the whole blood, or a plurality of such primer pairs are added to the reaction mixture.

36. The method of claim 25, wherein the reaction mixture, the primer pair, and the detection reagent, are contained by a lateral flow strip.
